(19) United States
(12) Patent Application Publication
(10) Pub. No.: US 2008/0274149 A1
(43) Pub. Date: Nov. 6, 2008

(54) Encapsulation and Controlled Release of Biologically Active Ingredients with Enzymatically Degradable Microparticulate, Hyperbranched Polymers

(75) Inventors: Matthias Seiler, Griesheim (DE); Saskia Klee, Essen (DE); Geoffrey Hills, Essen (DE); Peter Lersch, Dinslaken (DE); Irina Smirnova, Erlangen (DE); Wolfgang Arlt, Erlangen (DE); Juri Tschernjaew, Aschaffenburg (DE); Axel Kobus, Langen (DE)

Correspondence Address: SCULLY SCOTT MURPHY & PRESSER, PC, 400 GARDEN CITY PLAZA, SUITE 300, GARDEN CITY, NY, 11530, US

(73) Assignee: EVONIK DEGUSSA GMBH, Essen (DE)

(21) Appl. No.: 12/091,125

(22) PCT Filed: Sep. 6, 2006
(86) PCT No.: PCT/EP06/08663

§ 371 Date: Apr. 22, 2008

(30) Foreign Application Priority Data
Oct. 25, 2005 (DE) 102005051342.5

(51) Int. Cl. A61K 8/11; A61K 9/14; A61K 31/195; A61K 38/43; A61K 36/00; A61K 38/02; A01P 1/00; A61P 43/00; A61K 36/06; A61K 36/02; A61K 31/56; A01N 25/28

(52) U.S. Cl. 424/401; 424/497; 514/565; 424/94.1; 424/725; 424/195.16; 424/195.17; 514/182; 424/408; 424/59; 514/2; 424/402

(57) ABSTRACT

The invention provides encapsulated, microparticulate active ingredient formulations for the controlled release of active ingredients on skin and skin appendages consisting of encapsulation material as casing and at least one enclosed biologically active ingredient as core, which is characterized in that enzymatically degradable organic hyperbranched polymers containing ester groups are used as encapsulation material.

The invention relates to cosmetic preparations comprising one or more active ingredients in a microencapsulation whose hyperbranched encapsulation material containing ester groups is degraded by enzymes on skin and skin appendages.

When it is a question of achieving and promising particular effects of cosmetic products, the ingredients are a central theme. The high standard of supplied ingredients and raw materials in cosmetic formulations is being continually broadened since consumers are interested in high-performance and effective products which can counteract the effects of ageing. In this connection, the interest of the cosmetics manufacturers is also directed to active ingredients which are able to revitalize the skin or to protect against the consequences of photoageing. Whereas in the past such substances served primarily for smoothing and moisturizing the skin, they are nowadays supplemented by a large number of different materials with a physiological effect. Examples thereof are vitamins, fruit acids and also ceramides. In this connection, the way in which such active ingredients are stabilized is also of increasing importance. In cosmetics, there is great interest in active ingredients which can be stably stored in aqueous or else in water-containing systems.

It is desirable, for the purpose of applying one or more cosmetic skin active ingredients and/or aroma substances and/or food supplements, to encapsulate these or to provide them with a coating. In particular, this measure is suitable for thermolabile, oxidation-sensitive substances and also readily volatile fragrances.

Encapsulations are useful when active ingredients are to be protected and made to keep for longer, if they are to penetrate well into the skin, be uniformly distributed and released in a controlled manner.

In this connection, with cosmetic applications, it must be ensured that uncontrolled penetration into the skin does not result. This can be the case for nanoscale particle sizes.

U.S. Pat. No. 6,379,683 describes nanoparticles comprising a lipophilic core that is enclosed in dendritic polymers. These nanocapsules are said to effectively shield the core against external influences. Following application, the nanocapsules penetrate the skin and release the active ingredient in the inner skin layers.

It is therefore advisable to apply microscale particles to the skin which can release the cosmetically active ingredient on the skin as a result of a certain, skin-specific release mechanism. In this connection, an additional effect may result on the penetration of the active ingredient if the polymer forms as polymer film on the skin. This leads to a microocclusive effect which can aid the active ingredients when penetrating into the skin.

The aim of a microencapsulation can therefore serve different purposes, such as that of the controlled release behaviour of an active ingredient (controlled release), the coating of liquid substances, a masking or protection of the core material, the reduction in the volatility, and the improvement in the compatibility with other substances, for example for compounding.

According to the invention, the term “microcapsules” is understood as meaning particles and aggregates which contain an inner space or core which is filled with a solid, gelled, liquid or gaseous medium and are enclosed (encapsulated) by a continuous casing of film-forming polymers. These particles preferably have small dimensions.

In addition, the microscopically small capsules can comprise one or more cores distributed in the continuous encapsulation material, consisting of one or more layers. The distribution of the material to be encapsulated can even go so far that a homogeneous mixture of encapsulation material and core material is formed, which is referred to as a matrix. Matrix systems are also known as microparticles.

The preparation of microcapsules has been described in detail in the literature of the prior art and is accessible by means of known reactive and nonreactive processes, such as solvent evaporization, precipitation processes, coacervation, interfacial polycondensation, high-pressure encapsulation processes etc.

Solvent evaporization is used for producing reservoir and matrix systems; these include, inter alia, spray-drying and drum-coating.

In the precipitation process, the polymeric wall material is dissolved in a water-miscible solvent and the active ingredient to be encapsulated is dispersed therein. The dispersion is then introduced into the continuous aqueous phase with intense thorough mixing.

Coacervation is understood as meaning the separation of a colloidal dispersion (liquid/liquid or solid/liquid) in a phase with a high content of liquid dispersed material (coacervate) and a phase with a low content, brought about by external influences.

In the interfacial polycondensation method, in contrast to the other microencapsulation processes used, such as solvent evaporization or coacervation, which use already prepared polymers as coating materials, the shell is formed from the corresponding monomers only in the course of the encapsulation process.

The prior art with regard to high-pressure encapsulation processes with compressed or supercritical fluids is described by Gamse et al. in Chemie Ingenieur Technik 77 (2005) 669-680 and by McHugh and Krukonis in “Supercritical Fluid Extraction: Principles and Practices”, Stoneham Ma. 1986.

Gamse et al. Fages et al. and Bungert et al. describe in particular the high-pressure processes which are suitable for preparing microparticles and microcapsules (Gamse et al., Chemie Ingenieur Technik 77 (2005) 669-680; Fages et al., Powder Technology 141 (2004) 219-226 and Bungert et al., Ind. Eng. Chem. Res., 37, (1997) 3208-3220). The best known processes for preparing particles using compressed gases are the GAS (Gas Anti-Solvent) process, the PCA (Precipitation with a Compressed fluid Antisolvent) process, the PGSS (Particles from Gas Saturated Solutions) process and the RESS (Rapid Expansion of Supercritical Solutions) process. These processes are discussed briefly below.

In the GAS process, a solution which comprises polymer, active ingredient and solvent is initially introduced into an autoclave at constant temperature and then supplied with a gas as non-solvent, so that the polymer and the active ingredient precipitate out as fine particles. In this case, thorough mixing of the solution/suspension using a stirrer is useful in order to prevent agglomeration of the particles.

During the precipitation, the active ingredient molecules can be incorporated into the polymer matrix or be present as core (reservoir), around which a polymeric coating has formed. A suspension then forms which can be separated off by filtration. By washing the particles in a supercritical medium (fluid), solvent residues can be extracted. Besides the possibility of operating the process at low and thus active-ingredient-kind temperatures, the influence on the kinetics of the phase conversion, i.e. the particle formation, plays an important role in particular. While the supersaturation can be controlled through the progress and the intensity of the addition of gas, the particle size distribution can also be chosen. In a first step, the phase separation is initiated, and crystallization nuclei form in the form of droplets of the resulting polymer-rich phase, the subsequent microparticles. What matters now is not allowing these droplets to co coalesce and grow, but ensuring as rapid an extraction as possible of the solvent from these drops. The particles are then produced with small diameters (Gamse et al., Chemie Ingenieur Technik 77 (2005) 669-680 and Bungert et al., Ind. Eng. Chem. Res., 37, (1997) 3208-3220). By varying these two steps in a targeted manner, it is possible to adjust particle distribution and particle size.

The PCA process or else SEDS (Solution Enhanced Dispersion by Supercritical Fluids) process optimizes the two limiting parameters of the GAS process, namely the pressure build-up rate as initiator for particle formation and mass transfer in order to remove the solvent from the drops (Gamse et al., Chemie Ingenieur Technik 77 (2005) 669-680; Fages et al., Powder Technology 141 (2004) 219-226 and Bungert et al., Ind. Eng. Chem. Res., 37, (1997) 3208-3220). The active ingredient polymer solution from the autoclave is hereby compressed and, in an injection nozzle, is brought into contact with the supercritical gas and atomized together in the precipitation unit. In a downstream washing operation, the solvent is removed from the particles with the supercritical fluid by extraction. By conveying solution and supercritical fluid together in the nozzle shortly before the spraying operation, it is possible, through the short contact time, to achieve a high pressure build-up rate. As already explained above, this results in high supersaturation of the polymer/active ingredient solution. In this way, it is possible to achieve homogeneous distributions, and small particle sizes since, following the initiated phase separation, as a result of the atomization, fine dispersion takes place, during which, as a result of the high specific surface area of the polymer solution drops, improved mass transfer of the solvent into the supercritical gas can take place. Through the supercritical spray-drying, displacement crystallization and crystallization by solvent vaporization are combined.

The PGSS process differs in principle from the high-pressure processes described previously since it makes do without an (often toxic) solvent for the polymer. As described by Weidner in WO 95/21688, Gamse et al. in Chemie Ingenieur Technik 77 (2005) 669-680, Fages et al. in Powder Technology 141 (2004) 219-226 and Bungert et al. in Ind. Eng. Chem. Res., 37, (1997) 3208-3220, in this process, the effect of lowering the glass transition temperature of a polymer by the supercritical fluid is utilized. The polymer is melted in the supercritical fluid and the active ingredient is dispersed in the solution. This also lowers the viscosity of the polymer melt. The polymer-gas melt with the dispersed active ingredient is decompressed in the precipitation unit via a nozzle, where additionally supercritical gas can also be supplied to the nozzle. As a consequence of lowering the temperature by the Joule-Thomson effect, the solution cools, and the polymer precipitates out as a fine powder. The particles can be separated off from the gas stream via a cyclone or a downstream electrofilter. In this way, the various size fractions can be separated. The active ingredient can be dispersed in the polymer matrix due to the melting of the polymer. Decompression in the nozzle produces fine, monodisperse particles.

The RESS process resembles the PGSS process since in this process too, no organic solvent is used. As described by Gamse et al. in Chemie Ingenieur Technik 77 (2005) 669-680, Fages et al. in Powder Technology 141 (2004) 219-226 and Bungert et al. in Ind. Eng. Chem. Res., 37, (1997) 3208-3220, the polymer is firstly dissolved in the high-pressure autoclave. The active ingredient is either likewise dissolved or dispersed via a stirrer. In the case of charged microparticles, homogeneous distribution of the active ingredient in the melt is of very great importance since ultimately the size of the active ingredient molecules is the decisive limitation for the size of the microparticles (Gamse et al., Chemie Ingenieur Technik 77 (2005) 669-680; Fages et al., Powder Technology 141 (2004) 219-226 and Bungert et al., Ind. Eng. Chem. Res., 37, (1997) 3208-3220). The supercritical solution is atomized in a precipitation unit at ambient pressure. Compared to the processes described above, supersaturation of the solution or of the droplets during decompression occurs at a much greater rate. As a result of the decompression, the density of the supercritical fluid and thus also the dissolving capacity drops to gas-typical values in a very short time. In this process, nucleation and mass transfer follow one another directly and are optimized many times over compared with the other processes (Gamse et al., Chemie Ingenieur Technik 77 (2005) 669-680; Fages et al., Powder Technology 141 (2004) 219-226 and Bungert et al., Ind. Eng. Chem. Res., 37, (1997) 3208-3220).

In cosmetic formulations for the treatment also of normal skin, but in particular of sensitive, irritated skin and very particularly in babycare, it is, however, for obvious reasons, often problematic or impossible to use such microencapsulated active ingredients.

Furthermore, in skincare, it has to be ensured that the microflora of the skin is not harmed by unsuitable additives, but retained and supported, i.e. to largely maintain the “natural” ambient conditions.

The human skin has a balanced microflora which is in dynamic equilibrium with the tissue (Holland, K. T., Bojar, R. A., Am. J. Clin. Dermatol., 2002, 3, 445-449). The microflora can thus be regarded as an integral constituent of the skin. The majority of the microorganisms lives on the surface of the skin and in the follicles. Through a number of mechanisms, the skin controls the fact that that microorganisms cannot spread indiscriminately and in particular a stop is put on pathogenic microorganisms.

The microorganisms of the microflora produce enzymes which they release to the surrounding area. Enzymes are biological catalysts which increase the rate of the reactions in the human body without themselves being changed as a result. These enzymes also serve to convert or degrade molecules located on the skin. Hydrolysis reactions are catalyzed by hydrolases which, depending on the substrate property, can be divided into lipases, proteases, esterases, glycosidases, phosphatases etc. Thus, for example, lipase degrades natural fat (triglycerides) on the skin and scalp into glycerol and free fatty acids. Likewise, molecules which are secreted with perspiration can be degraded and, in so doing, produce an unpleasant smell of sweat.

It has also been shown that polymers with hydrolytically degradable structures can be degraded more quickly through the action of enzymes (Santerre, J. P et al., Biodegradation evaluation and polyester-urethanes with oxidative and hydrolytic enzymes, J. Biomed. Mater. Res., 28, 1187, 1997). Some enzymes are very specific and only cleave certain substrates. However, there are also nonspecific enzymes which can also cleave synthetic polymers, such as, for example, lipases.

Requirements which are ideally placed on an encapsulation system for cosmetic active ingredients are therefore manifold. Besides a gentle and rapid enclosure process, which should be easy to carry out and be suitable for preparing microcapsules of consistent quality, the active ingredient to be encapsulated should be enclosed as completely as possible because only then is protection adequate. Preferably, the preparation of the microcapsules takes place in a simple one-step process and uses, as wall material, commercially available polymers which are characterized by a defined chemical composition. Furthermore, when choosing the polymer material, it should be taken into consideration that no undesired skin reactions are triggered and that the type of release mechanism can be adjusted so that the microflora is not adversely affected.

An object of the present invention was to provide storage-stable and transport-stable cosmetic preparations for the treatment of the skin which comprise the active ingredients in a microencapsulation which satisfy a broad diversity of the requirement criteria already mentioned and, following application to the skin, release the active ingredient continuously and in a controlled manner on the skin without adversely affecting the microflora of the skin.

The invention thus provides encapsulated microparticulate active ingredient formulations for the controlled release of active ingredients on skin and skin appendages consisting of encapsulation material as casing and at least one enclosed biologically active ingredient, which is characterized in that enzymatically degradable organic hyperbranched polymers containing ester groups are used as encapsulation material.

The controlled release through endogenous enzymes of human or skin microflora origin does not have to take place here directly on the skin/the skin appendages. It is also possible on surface-treated textiles close to the skin by transferring these enzymes to these textiles.

This invention therefore further provides encapsulated, microparticulate active ingredient formulations for the controlled release of active ingredients on textiles consisting of encapsulation material as casing, which includes at least one especially biologically active ingredient, which is characterized in that enzymatically degradable organic hyperbranched polymers containing ester groups are used as encapsulation material.

Further subject matters of the invention are characterized by the claims.

Here, particular advantages are offered by a polymer system in which cosmetic active ingredients have only low solubility since, in such a polymer mixture, the active ingredient highly endeavours to leave the polymer. The low density of the system additionally ensures short diffusion routes.

Surprisingly, it has been found that by using hyper-branched macromolecules containing ester groups, it is possible to prepare microcapsules for incorporation into cosmetic formulations which can be produced without the use of additional agents and carrier materials and without the application of mechanical energy for making the capsule wall material permeable.

Highly branched, globular polymers are also referred to in the specialist literature as “dendritic polymers”. These dendritic polymers synthesized from multifunctional monomers can be divided into two different categories, the “dendrimers”, and the “hyperbranched polymers”.

Dendrimers have a very regular, radially symmetrical generation structure. They are monodisperse, globular polymers which—compared to hyperbranched polymers—are produced in multistage syntheses with high synthesis expenditure.

Here, the structure is characterized by three different areas:
(1) the polyfunctional core, which is the symmetry centre,
(2) various defined radially symmetrical layers of a repeat unit (generation) and
(3) the terminal groups.

In contrast to the dendrimers, the hyperbranched polymers are polydisperse and irregular as regards their branching and structure. Besides the dendritic units—in contrast to dendrimers—linear units also occur in hyperbranched polymers. One example of a hyperbranched polymer is shown in the structure below:

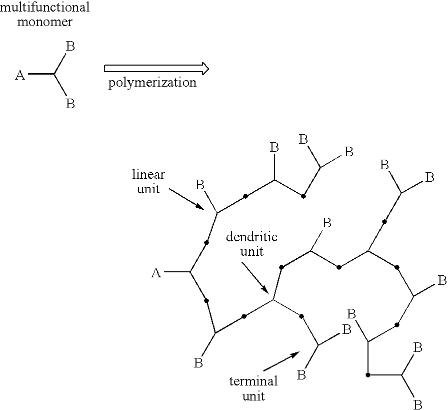

### Hyperbranched Polymer

As regards the different possibilities for the synthesis and the structure of hyperbranched polymers, reference may be made to a) Jikei M., Kakimoto M., Hyperbranched polymers: a promising new class of materials, Prog. Polym. Sci., 26 (2001) 1233-1285 and/or b) Gao C., Yan D., Hyperbranched Polymers: from synthesis to applications, Prog. Polym. Sci., 29 (2004) 183-275, c) Seiler, Fortschritt-Berichte VDI, series 3, No. 820 ISBN 3-18-382003-x, which are hereby incorporated by reference and serve as part of the disclosure of the present invention.

The hyperbranched and highly branched polymers containing ester groups described in these publications are also suitable for the purposes of the present invention for the encapsulation of active ingredients, referred to below as carrier polymers or encapsulation or coating material.

For the purposes of the present invention, the term “hyperbranched polymers” includes both dendrimers and also highly branched polymers.

The microcapsules prepared from these polymers by the known processes can vary as regards shape and size within a wide range depending on the preparation process, although they are preferably approximately globular or spherical and, depending on the substances present inside them, have a diameter in the range from 1 to 1000 μm, in particular from 5 to 200 μm and preferably from 10 to 50 μm. Some of the processes for the preparation of microcapsules are not suitable for the encapsulation of cosmetic active ingredients on account of their drastic preparation conditions with reaction temperatures above 100° C. since often under such conditions, the active ingredient to be encapsulated is for the greatest part decomposed or, in unfavourable cases, is even completely decomposed.

The release of the substances from the microcapsules is initiated by enzymatic action after application of the microcapsules-containing preparation. Hereby, the enzymatic action degrades the encapsulation material.

It has also been established that also by mixing a hyperbranched base polymer containing ester groups with any other desired polymers, the enzymatically initiated release behaviour is retained if the amount of hyper branched base polymer constitutes more than 70% by weight. By mixing with other polymers, preferably polymers functionalized with ionizable groups, it is possible to favourably influence properties such as biodegradability, the release behaviour of the active ingredients and also the production costs.

In a preferred embodiment of the invention, the cosmetic preparations comprise microcapsules in amounts of from 0.1 to 10% by weight, in particular 0.2 to 8% by weight, particularly preferably 0.5 to 5% by weight.

The encapsulation materials used according to the invention are hyperbranched polyesters based on a molar mass between 1000 g/mol and 100 000 g/mol, preferably between 1500 g/mol and 70 000 g/mol and particularly preferably between 4000 g/mol and 50 000 g/mol, in which the bonding units have at least two bonding possibilities. Hyperbranched carrier polymers preferred in this connection are polyesters and polyester amides. Among these polymers, preference is given to the hyper-branched polyesters already commercially available under the name Boltorn® from Perstorp AB, and in particular also hyperbranched Boltorn® polyesters which are completely or partially esterified with fatty acids, preferably to 1 to 99%, in particular to 30 to 98%, and also the hyperbranched polyester amides available under the name Hybrane® from DSM BV Niederlande.

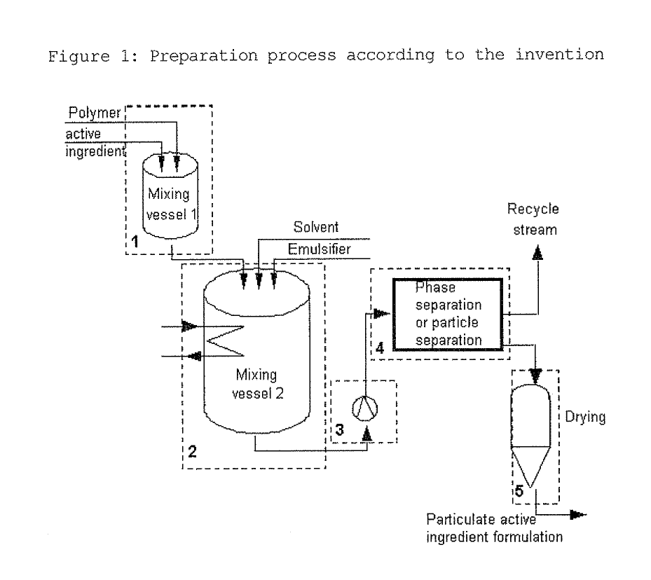

Surprisingly, it has been found that the particularly preferred encapsulation process described in FIG. 1 can be used without organic solvents for the encapsulation of active ingredients. The hyperbranched polymer functions here itself as solvent or dispersant. The unnecessary use of solvent or gas concentrations as a result leads to safer processes compared with the prior art since the hyperbranched polymers according to the invention are unable to form any explosive vapours like other solvents of the prior art.

The active ingredient formulations according to the invention comprise a hyperbranched polymer with a hydrophilic core. Hydrophilic means that the core is able to absorb a high fraction of water. According to a preferred aspect of the present invention, the hydrophilic core is soluble in water. Preferably, the solubility in water at 90° C. is at least 7 mass percent, particularly preferably at least 20 mass percent. This parameter is measured using the hyperbranched polymer before the hydrophobicization, i.e. on the hydrophilic core as such. The measurement can take place according to the so-called flask method, where the solubility of the pure substance in water is measured. In this method, the substance (solids have to be pulverized) is dissolved in water at a temperature slightly above the test temperature. When saturation is reached, the solution is cooled and kept at the test temperature. The solution is stirred until equilibrium is reached. Alternatively, the measurement can be carried out directly at the test temperature if, by taking an appropriate sample, it is ensured that the saturation equilibrium has been reached. The concentration of the test substance in the aqueous solution, which must not contain any undissolved substance particles, is then determined using a suitable analytical method.

Besides the hydrophilic core, the hyperbranched polymer has hydrophobic end groups. In this connection, the term hydrophobic end groups means that at least some of the chain ends of the hyperbranched polymer have hydrophobic groups. It may be assumed here that, as a result of this, an at least partially hydrophobicized surface is obtained.

The term hydrophobic is known per se in the specialist world, where the groups which are present at least in some of the ends of the hyperbranched polymers, when considered by themselves, have a low solubility in water.

According to one particular aspect, the surface is hydrophobicized by groups which are derived from carboxylic acids having at least 6, preferably at least 12, carbon atoms. The carboxylic acids have preferably at most 40, particularly at most 32, carbon atoms. Here, the groups can be derived from saturated and/or unsaturated fatty acids.

These include in particular fatty acids which are present in linseed, soybeans and/or tall oil. Of particular suitability are fatty acids which have a low fraction of double bonds.

It is possible to use the monobasic fatty acids based on natural vegetable or animal fats and oils having 6 to 22 carbon atoms, in particular having 14 to 18 carbon atoms, that are customary and known in this field, such as caproic acid, caprylic acid, capric acid, lauric acid, myristic acid, palmitic acid, palmitoleic acid, isostearic acid, stearic acid, oleic acid, linoleic acid, petroselinic acid, elaidic acid, arachic acid, behenic acid, erucic acid, gadoleic acid, rapeseed oil fatty acid, soybean oil fatty acid, sunflower oil fatty acid, tall oil fatty acid, which can be used on their own or in a mixture in the form of their glycerides, methyl or ethyl esters or as free acids, and also the technical-grade mixtures that are produced during the pressurized cleavage. Of suitability in principle are all fatty acids with a similar chain distribution.

The content of unsaturated fractions in these fatty acids and fatty acid esters is—where this is required—adjusted through the known catalytic hydrogenation processes to a desired iodine number or achieved by mixing completely hydrogenated fatty components with unhydrogenated fatty components.

The iodine number, being a measure of the average degree of saturation of a fatty acid, is the amount of iodine which is taken up by 100 g of the compound to saturate the double bonds.

Preferred carboxylic acids here have a melting point of at least 35° C. and preferably at least 40° C. Accordingly, linear, saturated carboxylic acids are preferably used. These include, in particular, dodecanoic acid, tetradecanoic acid, hexadecanoic acid, heptadecanoic acid, octadecanoic acid, eicosanoic acid, docosanoic acid and tetracosanoic acid. Particular preference is given to saturated fatty acids having 16 to 22 carbon atoms.

The hyperbranched carrier polymer (after the hydrophobicization) has a molecular weight of at least 1500 g/mol. Preferably, the molecular weight is at most 100 000 g/mol, particularly preferably at most 50 000 g/mol. This parameter refers to the weight-average of the molecular weight (Mw), which can be measured by means of gel permeation chromatography, the measurement being carried out in DMF and the reference used being polyethylene glycols (cf. inter alia Burgath et. al in Macromol. Chem. Phys., 201 (2000) 782-791). Use is made here of a calibration curve which has been obtained using polystyrene standards. This parameter is therefore an apparent measurement.

The polydispersity Mw/Mn of preferred hyperbranched polymers is preferably in the range from 1.01 to 6.0, particularly preferably in the range from 1.10 to 5.0 and very particularly preferably in the range from 1.2 to 3.0, where the number average of the molecular weight (Mn) can likewise be obtained by GPC.

The viscosity of the hyperbranched polymer is preferably in the range from 50 mPas to 5.00 Pas, particularly preferably in the range from 70 mPas to 3.00 Pas, it being possible to measure this parameter by means of rotation viscometry at 110° C. and 30 s⁻¹ between two 20 mm plates.

The acid number of the hyperbranched polymer is preferably in the range from 0 to 20 mg KOH/g, particularly preferably in the range from 1 to 15 mg KOH/g and very particularly preferably in the range from 6 to 10 mg KOH/g. This property can be measured by titration with NaOH (cf. DIN 53402).

Furthermore, after the hydrophobicization, the hyperbranched polymer has a hydroxy value in the range from 0 to 600 mg KOH/g, preferably from 0 to 300 mg KOH/g and particularly preferably in the range from 0 to 200 mg KOH/g. This property is measured according to ASTM E222. Here, the polymer is reacted with a defined amount of acetic anhydride. Unreacted acetic anhydride is hydrolyzed with water. The mixture is then titrated with NaOH. The hydroxy value results from the difference between a comparison sample and the value measured for the polymer. In this connection, the number of acid groups in the polymer is to be taken into consideration. This can take place through the acid number, which can be determined using the process described above.

The degree of branching in the hyperbranched polymer is in the range from 1 to 99%, preferably 30 to 98%. The degree of branching is dependent on the components used for preparing the polymer, in particular the hydrophilic core, and also on the reaction conditions. The degree of branching can be determined according to Frey et al., this process being explained in D. Hölter, A. Burgath, H. Frey, Acta Polymer, 1997, 48, 30 and H. Magnusson, E. Malmström, A. Hult, M. Joansson, Polymer 2002, 43, 301.

The hyperbranched polymer has a melting temperature of at least 30° C., particularly preferably at least 35° C. and very particularly preferably at least 40° C. The melting temperature can take place by means of differential scanning calorimetry (DSC), e.g. using the Mettler DSC 27 HP apparatus and a heating rate of 10° C./min.

The solubility in water of the hyperbranched polymer after the hydrophobicization is preferably at most 10 mass percent, particularly preferably at most 7 mass percent and very particularly preferably at most 5 mass percent, measured according to the flask method explained above at 40° C.

Encapsulation materials that can be co-used according to the invention are the natural, semisynthetic or synthetic, inorganic and in particular organic materials known in the prior art, provided it is ensured that the enzymatically controlled opening of the resulting mixtures is retained.

Natural organic materials are, for example, homo- and heteropolymers of carbohydrates, amino acids, nucleic acids, amides, glucosamines, esters, gum arabic, agar agar, agarose, maltodextrins, alginic acid and its salts, e.g. sodium alginate or calcium alginate, liposomes, fats and fatty acids, cetyl alcohol, collagen, chitosan, lecithins, gelatin, albumin, shellac, polysaccharides, such as starch or dextran, cyclodextrins, sucrose and waxes.

Semisynthetic encapsulation materials are, inter alia, chemically modified celluloses, in particular cellulose esters and ethers, e.g. cellulose acetate, ethyl cellulose, hydroxypropylcellulose, hydroxypropylmethylcellulose and carboxymethylcellulose, and also starch derivatives, in particular starch ethers and esters.

Synthetic encapsulation materials are, for example, polymers, such as amino resins, polyacrylates, polyamides, polyvinyl alcohol or polyvinylpyrrolidone, organopolysiloxanes, non-natural amino acids, non-natural nucleic acids, polyamines, polyols, oligo- and polyisoprenes, esters and polyesters, in particular branched glycerol esters, amides, imines, polyphenols, dithiols and phosphodiesters, ethylene glycol, oxymethylene glycoside, acetal units, silicates and carbonates, hyperbranched hydrogels, comb polymers with polyester structure or polyvinylpyrrolidone, polylactide.

Furthermore, preferred carrier polymers that can be co-used are polycaprolactones, copolymers such as poly(D,L-lactide-co-glycolides), and the polyester compounds manufactured by Degussa AG from the product families Dynapol®S and Dynacoll®. These polymers can also serve as admixture for adjusting specific polymer properties.

By admixing these polyesters, the composition of the polymer can be adjusted such that the resulting encapsulation material can be sooner or later enzymatically degraded.

Typical examples of active ingredients as are used in the field of cosmetic preparations are vitamins, vitamin derivatives and complexes, enzymes, surfactants, cosmetic oils, pearlescent waxes, stabilizers, antimicrobial active ingredients, antiinflammatory active ingredients, plant, yeast and algae extracts, synthetic natural substances, amino acids and amino acid derivatives, such as creatine, bioactive lipids, such as cholesterol, ceramides and pseudoceramides, deodorants, antiperspirants, antidandruff agents, UV sun protection factors, antioxidants, preservatives, insect repellants, self-tanning agents, tyrosinase inhibitors (depigmentation agents), perfume oils, dyes, peroxides, peptides, oligopeptides or fragrances. Active ingredients preferably used are those which, in non-encapsulated form, can either not be stably incorporated into formulations or at least do not remain stable over prolonged storage periods. One example of a particularly preferred active ingredient is creatine.

The cosmetic preparations for the treatment of the skin are formulations customary in practice which comprise typical constituents for the particular intended uses in the customary amounts. These formulations are known to the person skilled in the art and can thus be used.

The currently used enzymatically degradable capsule systems are based on natural polymers (chitosan, alginates, gelatin, etc.) which swell in the presence of water. As a result of the water diffusing in from the surrounding area, on the one hand, the encapsulated active ingredient inside the capsules is attacked and, on the other hand, the active ingredient is released from the inside of the capsules into the surrounding area. There is no adequate protection of the active ingredient here.

The active ingredient microcapsules prepared in the present invention from hyperbranched polymers esterified with fatty acids can prevent the penetration of water through their hydrophobic surface modification and thus offer better protection of the active ingredient. Furthermore, enzymatic degradation of the carrier polymer is improved compared to the natural polymers, which brings with it more rapid and more efficient release of the active ingredient.

Compared to current active ingredient-containing microcapsules, the microparticulate active ingredient formulations according to the invention—prepared by the encapsulation processes described above and in FIG. 1—have a further advantage: stability to shear forces. Since the active ingredient-containing microcapsules and the formulations thereof are currently supplied in part as dispersions, they must be incorporated into a cream formulation at the end of processing and under mild conditions due to the swollen nature of the material. This is unnecessary in the case of the active ingredient-containing microcapsules used according to the invention since they have a solid structure which is stable to shear forces.

Moreover, the active ingredient particles particularly suitable according to the invention have a particle size of, on average, 10 to 60 μm and release the encapsulated cosmetic ingredient into the surrounding area of the active ingredient formulation to at least 30% by weight within 24 h, preferably within 15 h and particularly preferably within 10 h.

The desired particle sizes and active ingredient loading concentrations can be produced by coacervation or preferably by active ingredient dispersion in a carrier polymer melt or a solution rich in carrier polymer in the temperature range between −30° C. and +150° C. and particularly preferably between 0° C. and +60° C. and a pressure range between 0.1 mbar and 250 bar and preferably between 1 mbar and 10 bar. Alternatively, generation of the active ingredient formulations according to the invention is also possible with spray-drying, the GAS (Gas AntiSolvent) process, the PCA (Precipitation with a Compressed fluid Antisolvent) process, the PGSS (Particles from Gas Saturated Solutions) process and the RESS (Rapid Expansion of Supercritical Solutions) process, however only in the temperature range between −30° C. and +150° C., preferably between 0° C. and +100° C., and at system pressures between 0.1 mbar and 250 bar, preferably between 1 bar and 180 bar.

The active ingredient formulations prepared using these processes according to the invention—using the above-described carrier polymers according to the invention—exhibit particularly high stability, as a result of which particularly sensitive, reactive or unstable cosmetic active ingredients can be processed to give more advantageous cosmetic formulations.

Based on the mass of the carrier polymer, the active ingredient formulations according to the invention are preferably characterized by active ingredient concentrations between about 0.5 and 90 mass %.

Furthermore, it has surprisingly been found that in the case of hyperbranched carrier polymers (on account of the comparatively low melting and solution viscosities for polymers), the encapsulation processes can be operated without solvents or compressed gases. The hyperbranched polymer can itself act as solvent/dispersant. The use of solvent/gas concentrations is not required as a result, leading to safer processes compared with the prior art since hyperbranched polymers cannot form explosive or hazardous vapours like other solvents of the prior art.

The examples below are intended to illustrate the subject matter of the invention in more detail:

### EXAMPLE 1

### Microencapsulation of Creatine

Using the process according to the invention shown in FIG. 1, creatine was encapsulated in a hyperbranched, enzymatically degradable polyester. For this, in mixing vessel 1 (see FIG. 1), 20% by weight of the commercially available biologically active ingredient creatine was dispersed in a polymer melt at T=85° C. by intense mixing in a stirred container for 0.5 minute. The polymer melt consisted of a molten hyperbranched, fatty-acid-modified polyester (M_{w}=7500 g/mol) which was obtained by esterifying 50% of the hydroxy groups of the hyperbranched polyester Boltorn H30, commercially available from Perstorp, with a mixture of stearic acid and palmitic acid (mass-based ratio of stearic acid to palmitic acid=2:1).

In mixing vessel 2 (see FIG. 1), a mixture of surfactants consisting of 2% by weight of polyvinyl alcohol (M=6000 g/mol) and 0.1% by weight of an ethoxylated fatty alcohol, Tego Alkanol L4, was initially introduced into water at 50° C. with stirring.

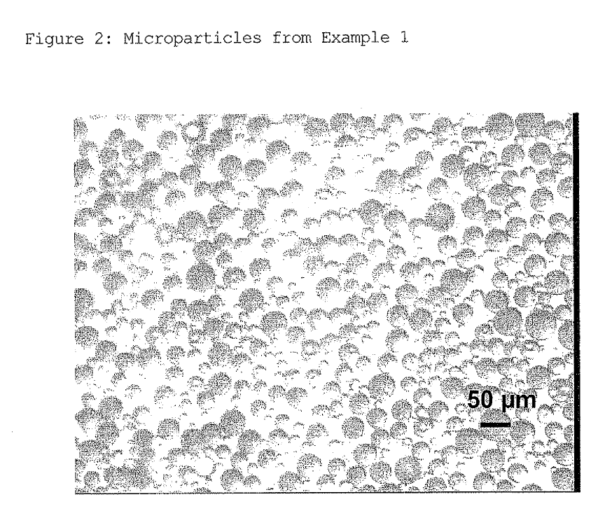

The polymer/creatine dispersion from mixing vessel 1 was then added to the external phase in mixing vessel 2 with continuous stirring using an ULTRA-TURRAX stirrer at 3000 revolutions per minute[ ]. After a residence time of 5 minutes and a reduction in the system temperature to a temperature which is 10° C. below the melting temperature of the polymer, solid particles are formed. These particles exhibit a particle size distribution of 10 μm<d_{90,particles}<50 μm (FIG. 2) and consist of the hyperbranched, fatty-acid-modified polyester which includes about 17% by weight of creatine (based on the particle mass). Using a peristaltic tube pump (system 3), the suspension was conveyed to a centrifuge (system 4), where, at 25° C., the microparticulate active ingredient formulation is separated off from the continuous phase. The microparticles were then dried in a vacuum dryer at 25° C. and 10 mbar for 100 h. The microparticles are thus present in free-flowing form and are spherical, meaning that negative sensory effects will not occur on the skin.

### EXAMPLE 2

### Microencapsulation of Tocopherol

Using the process according to the invention shown in FIG. 1, tocopherol was encapsulated in a hyperbranched, enzymatically degradable polyester. For this, in mixing vessel 1 (see FIG. 1), 20% by weight of the biologically active ingredient tocopherol was dispersed in a polymer melt at T=85° C. by intense mixing (e.g. in a stirred container using an anchor stirrer at 100 revolutions per minute) for 0.5 minute. The polymer melt consisted of a molten hyperbranched, fatty-acid-modified polyester (M_{w}=about 10 000 g/mol) which was obtained by esterifying 90% of the hydroxy groups of the hyperbranched polyester Boltorn H30, commercially available from Perstorp, with a mixture of stearic acid and palmitic acid (mass-based ratio of stearic acid to palmitic acid=2:1).

In mixing vessel 2 (see FIG. 1), a mixture of surfactants consisting of 2% by weight of polyvinyl alcohol (M 6000 g/mol) and 0.1% by weight of an ethoxylated fatty alcohol, Tego Alkanol L4, was initially introduced into water at 50° C. with stirring. This mixture functions as continuous phase.

The polymer/active ingredient dispersion from mixing vessel 1 was then added to the external phase in mixing vessel 2 with continuous stirring using an ULTRA-TURRAX stirrer at 3000 rpm. After a residence time of 5 minutes and a reduction in the system temperature to a temperature which is 10° C. below the melting temperature of the polymer, solid particles are formed. These particles exhibit a particle size distribution of 10 μm<d_{90,particles}<60 μm and consist of the hyperbranched fatty-acid-modified polyester which includes about 16% by weight of tocopherol (based on the particle mass) Using a peristaltic tube pump (system 3), the suspension was conveyed to a centrifuge (system 4), where, at 25° C., the active ingredient particles are separated off from the continuous phase. The active ingredient particles were then dried in a vacuum dryer at 25° C. and 10 mbar for 100 h. The microparticles are thus present in free-flowing form and are spherical, meaning that negative sensory effects will not occur on the skin.

It could be shown for the first time that the active ingredient particles according to the invention prepared in this way have a combination of properties that is extremely advantageous for cosmetic applications:
(i) Release of tocopherol by enzymatic degradation of the carrier polymer
   0.22 g of tocopherol-loaded polymer particles were suspended in 15 ml of phosphate buffer, pH 5.0, or in 15 ml solution of the lipase from Candida cylindracea, 0.5 mg/ml, in the same buffer at 37° C. More than 60% by weight of the encapsulated tocopherol was released in this release experiment in the presence of lipase by enzymatic degradation of the carrier polymer after 12 h. Whereas in the enzyme-free buffer solution, less than 10% by weight of tocopherol was released after 12 h.
(ii) Encapsulation efficiency of tocopherol
   Using the process according to the invention shown in FIG. 1, it was possible to encapsulate about 80% by weight of the tocopherol used (initially introduced in mixing vessel 1). This was determined by analysis of the active ingredient particles with UV-vis analysis (Perkin Elmar UV-vis instrument “Lamda 650%”) and comparison with the tocopherol concentration used.
(iii) Tocopherol content of the active ingredient particles
   As could be shown with UV-vis analysis (Perkin Elmar UV-vis instrument “Lamda 650”), it was possible, using the described procedure, to achieve a concentration of about 16% by weight of tocopherol which is suitable for cosmetic applications.
(iv) Shear stability
   The microparticles prepared according to FIG. 1 were incorporated into a cream formulation using an ULTRA-TURRAX stirrer, which was stirred for 1 minute at 15 000 revolutions per minute. A comparison of the micrographs of the active ingredient particles before and after the incorporation into an oil phase (Parrafin WINOG20 Pharma, Univar GmbH) showed that no change in the particle integrity can be seen.
(v) Solvent-free microencapsulation process with solvent-free particles
   Surprisingly, it was found for the first time that in the case of hyperbranched carrier polymers, the encapsulation process described in FIG. 1 can be operated without organic solvents and be used for the tocopherol encapsulation. The hyperbranched, fatty-acid-modified polymer can itself function as solvent and/or dispersant. The solvent concentrations reduced as a result lead to safer and more sustainable processes compared to the prior art since the hyperbranched polymers according to the invention cannot form any explosive vapours like other solvents of the prior art. In contrast to the prior art, the prepared polymer/tocopherol particles are free from organic solvent, which could be shown by analysis of the active ingredient particles using headspace gas chromatography (Agilent HP 7694 in combination with Agilent GC 6890) according to the descriptions by Hachenberg and Beringer in “Die Headspace-Gaschromatographie als Analysen- und Meβmethode” [Headspace gas chromatography as analytical and measurement method], Vieweg Verlag, Braunschweig, Wiesbaden, 1996.

### EXAMPLE 3

### Microencapsulation of Folic Acid

Using the process according to the invention shown in FIG. 1, folic acid was encapsulated in a hyperbranched, enzymatically degradable polyester. For this, in mixing vessel 1 (see FIG. 1), 20% by weight of folic acid was dispersed in a polymer melt at T=85° C. by intense mixing (in a stirred container using an anchor stirrer at 100 revolutions per minute). The polymer melt consisted of a molten hyperbranched, fatty-acid-modified polyester (M=about 10 000 g/mol) which was obtained by esterifying 50% of the hydroxy groups of the hyperbranched polyester Boltorn H30, commercially available from Perstorp, with a mixture of stearic acid and palmitic acid (mass-based ratio of stearic acid to palmitic acid=2:1).

In mixing vessel 2 (see FIG. 1), a mixture of surfactants consisting of 2% by weight of polyvinyl alcohol (M=6000 g/mol) and 0.1% by weight of an ethoxylated fatty alcohol, Tego Alkanol L4, was initially introduced into water at 50° C. with stirring. This mixture functions as continuous phase.

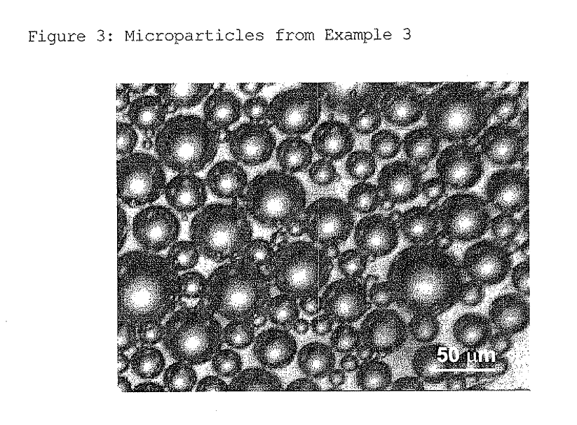

The polymer/active ingredient dispersion from mixing vessel 1 was then added to the external phase in mixing vessel 2 with continuous stirring using an ULTRA-TURRAX stirrer at 3000 revolutions per minute. After a residence time of 5 minutes and a reduction in the system temperature to a temperature which is 10° C. below the melting temperature of the polymer, particles are formed. These particles exhibit a particle size distribution of 10 μm<d_{90,particle}<60 μM (see FIG. 3) and consist of the hyperbranched, fatty-acid-modified polyester which includes about 18% by weight of folic acid (based on the particle mass). Using a peristaltic tube pump (system 3), the suspension was conveyed to a centrifuge (system 4), where, at 25° C., the active ingredient particles are separated off from the continuous phase. The active ingredient particles were then dried in a vacuum dryer at 25° C. and 10 mbar for 100 h. The microparticles are thus present in free-flowing form and are spherical, meaning that negative sensory effects will not occur on the skin.

It could be shown for the first time that the active ingredient particles according to the invention prepared in this way have a combination of properties that is extremely advantageous for cosmetic applications:
(i) Release of folic acid through enzymatic degradation of the carrier polymer
   0.22 g of folic acid-loaded polymer particles were suspended in 15 ml of phosphate buffer, pH 5.0, or in 15 ml solution of the lipase from Candida cylindracea, 0.5 mg/ml, in the same buffer at 370C. More than 70% by weight of the encapsulated folic acid was released in this release experiment in the presence of lipase by enzymatic degradation of the carrier polymer after 12 h. Whereas in the enzyme-free buffer solution, less than 10% by weight of folic acid was released after 12 h.
(ii) Encapsulation efficiency of folic acid
   Using the process according to the invention shown in FIG. 1, it was possible to encapsulate about 90% by weight of the folic acid used (initially introduced in mixing vessel 1). This was determined by analysis of the active ingredient particles with UV-vis analysis (Perkin Elmar UV-vis instrument “Lamda 650”) and comparison with the folic acid concentration used.
(iii) Folic acid content of the active ingredient particles
   As could be shown with UV-vis analysis (Perkin Elmar UV-vis instrument “Lamda 650”), it was possible, using the described procedure, to achieve a concentration of about 18% by weight of folic acid suitable for cosmetic applications.
(iv) Shear stability
   The microparticles prepared according to FIG. 1 were incorporated into an oil phase (Parrafin WINOG20 Pharma, Univar GmbH) using an ULTRA-TURRAX stirrer, which stirred for 1 minute at 15 000 revolutions per minute. A comparison of the micrographs of the active ingredient particles before and after the incorporation into the cream formulation showed that no change in the particle integrity is to be seen.
(v) Solvent-free microencapsulation process with solvent-free particles
   Surprisingly, it was found for the first time that in the case of hyperbranched carrier polymers, the encapsulation process described in FIG. 1 can be operated without organic solvents and can be used for the folio acid encapsulation. The hyper-branched, fatty-acid-modified polymer can itself function as solvent and/or dispersant. The solvent concentrations reduced as a result lead to safer and more sustainable processes compared to the prior art since the hyperbranched polymers according to the invention cannot form any explosive vapours like other solvents of the prior art. In contrast to the prior art, the prepared polymer/folic acid particles are free from organic solvent, which could be shown by analysis of the active ingredient particles using headspace gas chromatography (Agilent HP 7694 in combination with Agilent GC 6890) according to the descriptions by Hachenberg and Beringer in “Die Headspace-Gaschromatographie als Analysen- und Meβmethode” [Headspace gas chromatography as analytical and measurement method], Vieweg Verlag, Braunschweig, Wiesbaden, 1996.

### EXAMPLE 4

### Microparticles from Polymer Blend

A hyperbranched, fatty-acid-modified polyester (M_{w}=7500 g/mol), which was obtained by esterifying 50% of the hydroxy groups of the commercially available hyper-branched polyester Boltorn H30 (Perstorp, Sweden) with a mixture of stearic acid and palmitic acid (mass-based ratio of stearic acid to palmitic acid=2:1), was melted together in the ratio 1:1 with a further hyperbranched, fatty-acid-modified polyester (M_{w}=10 500 g/mol), which was obtained by esterifying 90% of the hydroxy groups of the commercially available hyperbranched polyester Boltorn H30 (Perstorp, Sweden) with a mixture of arachic acid and behenic acid (mass-based ratio of arachic acid to behenic acid=2:3), to prepare a homogeneous polymer blend. The polymer blend prepared in this way was then melted further—as described in Example 1.

The biologically active ingredient creatine was added to mixing vessel 1 (20% by weight) and dispersed in the polymer melt by intense mixing. In mixing vessel 2, a mixture of surfactants consisting of 2% by weight of polyvinyl alcohol (M=6000 g/mol) and 0.1% by weight of an ethoxylated fatty alcohol, Tego Alkanol L4, was initially introduced into water at 50° C. with stirring. This mixture functions as continuous phase.

The polymer/active ingredient dispersion from mixing vessel 1 was then added to the external phase in mixing vessel 2 with continuous stirring using an ULTRA TURRAX stirrer at 3000 rpm. After a residence time of 5 minutes and a reduction in the system temperature to a temperature which is 10° C. below the melting temperature of the polymer, particles are formed. These microparticles exhibit a particle size distribution as described in Example 1, and contain about 12% by weight of creatine. The microparticles are thus present in free-flowing form and are spherical, meaning that negative sensory effects will occur on the skin.

It could be shown for the first time that the active ingredient particles according to the invention prepared in this way are extremely advantageous for cosmetic applications:

The release of creatine was investigated as in Example 2 (i). It was found that the amount of released creatine in the presence of lipase after 22 hours was 45% by weight, based on the original creatine content.

Without lipase, about 10% was released.

### EXAMPLE 5

### Stability in Formulation

1.5% of the microcapsules from Example 4 were stirred into an oil-in-water emulsion and stored at T=45° C. After 5 weeks, the analytical determination showed that about 35% more creatine was stabilized by the encapsulation than in the case of free creatine. Rearrangement of creatine to inactive creatinine is thus reduced.

| | 0 weeks | 5 weeks |
|---|---|---|
| Free creatine [%] | 100 | 40 |
| Encapsulated creatine | 100 | 53.80 |
| [%] | | |
| Stabilization [%] | | 34.5 |

### EXAMPLE 6

### Demonstration of the Enzymatic Degradation of Boltorn H30 and Boltorn H40

The degradation of the molecules Boltorn H30 and Boltorn H40 (Perstorp) in aqueous, enzyme-containing solutions was shown by the following experiments:

The polymers Boltorn H30 and Boltorn H40 (Perstorp) were ground in separate experiments in an electric mill and sieved. Processing was further carried out with the fraction 90 μm<d_{particles}<250 μm. The polymer particles were suspended in a solution of lipase from Candida cylindracea, 0.5 mg/ml and phosphate buffer, pH=5, at 37° C. The control sample used was pure buffer under the same conditions. The concentration of the monomer 2,2-bishydroxymethylpropionic acid in the hyperbranched polymers Boltorn H30 and Boltorn H40 was analysed using UV spectroscopy (peak at 208.5 nm).

The concentration of the hydroxymethylpropionic acid in the lipase-containing solution after 24 h is greater by a factor of 4.7 than the concentration in pure buffer. The polymer Boltorn H30 is thus an enzymatically degradable, hyperbranched polymer.

### Bottom H30 Experiments:

Concentration of hydroxymethylpropionic acid (g/ml)

| Time (hours) | Buffer | Buffer/lipase |
|---|---|---|
| 0 | 0.045 | 0.045 |
| 24 | 0.088 | 0.248 |

The concentration of the hydroxymethylpropionic acid in the lipase-containing solution after 24 hours is greater by a factor of 4.8 than the concentration in pure buffer. The polymer Boltorn H40 is thus an enzymatically degradable, hyperbranched polymer.

### Boltorn H40 Experiments:

Concentration of hydroxymethylpropionic acid (g/ml)

| Time (hours) | Buffer | Buffer/lipase |
|---|---|---|
| 0 | 0.047 | 0.047 |
| 24 | 0.093 | 0.270 |

### EXAMPLE 7

### Fatty Acid Degradation

The described hyperbranched polyester from Example 1 was finely ground and 0.5 g of the powder were incubated in 50 ml of buffer, pH 7, at 37° C. with 5 mg of the lipase from Candida cylindracea. In the GC analysis, 1.6% of the fatty acid were released after 30 minutes. Without lipase, no hydrolysis took place

### EXAMPLE 8

Corresponding to Example 2 (i), the microparticles loaded with creatine from Example 1 were incubated with a lipase from Mucor miehei. After 24 hours, in the presence of lipase, 70% by weight of the creatine, based on the original creatine content, were released. By contrast, less than 20% by weight of creatine was released in the enzyme-free buffer solution.

### EXAMPLE 9

### Particle Integrity in the Formulation

Microparticles from Example 1 were added to an oil-in-water emulsion and viewed microscopically at 45° C. over time. In contrast to other polymers, which swell as a result of water diffusing in, such as, for example, gelatin or alginate, it is found that, at 45° C., after at least 12 weeks, no change in the shape and size of the microparticles is to be observed.

### EXAMPLE 10

### Shear Stability of the Microparticles

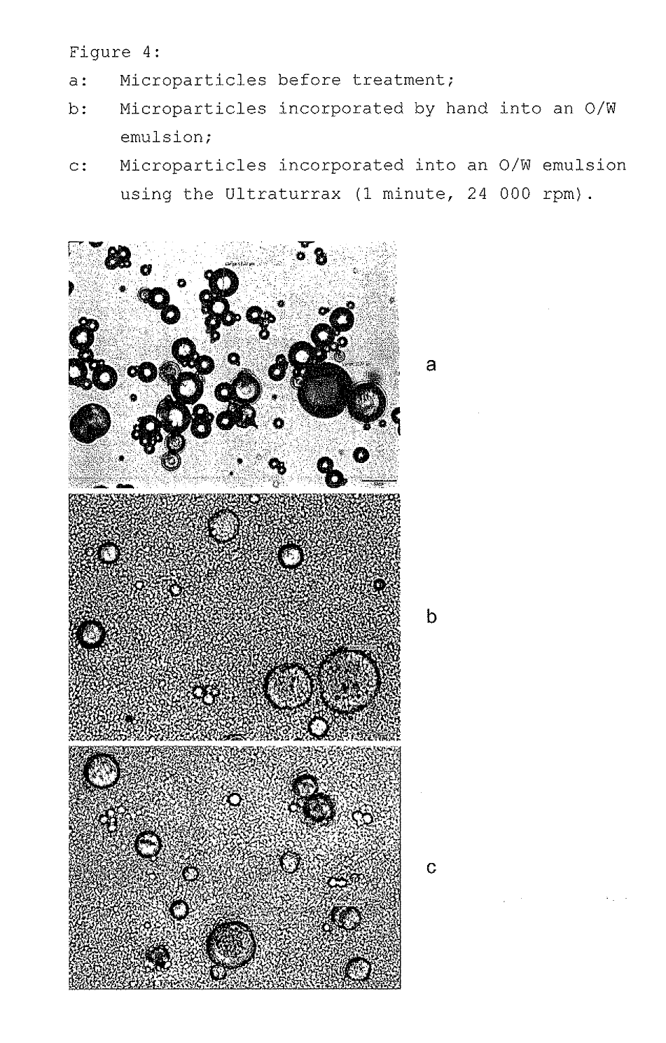

During the homogeneous incorporation of microparticles into a cream formulation, very high shear forces can sometimes act upon the microparticles. In this connection, the microparticles may be destroyed. For this reason, microparticles from Example 1 were viewed before and after incorporation into a cream formulation using an Ultraturrax (1 minute, 24 000 rpm). FIG. 4 shows that, after the treatment, no change in the particle integrity is to be seen.

1. Encapsulated, microparticulate active ingredient formulations for the controlled release of active ingredients on skin and skin appendages consisting of encapsulation material as casing and at least one enclosed biologically active ingredient, wherein enzymatically degradable organic hyperbranched polymers containing ester groups are used as encapsulation material.

2. Encapsulated active ingredient formulation according to claim 1, wherein the encapsulation material is a hyperbranched polymer with a molar mass between 1000 and 70 000 g/μmol, a melting temperature of at least 30° C. and a hydroxy value between 0 mg KOH/g and 600 mg KOH/g.

3. Encapsulated active ingredient formulation according to claim 1, wherein hyperbranched polyesters esterified completely or partially with fatty acids and having a molar mass between 1500 and 100 000 g/mol, a melting temperature of at least 20° C., a water solubility at 40° C. of less than 5 mass %, and a hydroxy value between 0 mg KOH/g and 600 mg KOH/g are used as encapsulation material.

4. Encapsulated active ingredient formulation according to claim 1, wherein the controlled release of the active ingredient is initiated by contacting the active ingredient formulation with at least one enzyme that is in the surroundings.

5. Encapsulated active ingredient formulation according to claim 1, wherein less than 30% by weight, based on hyperbranched base polymer, of known encapsulation materials are used as additional fractions of encapsulation materials.

6. Encapsulated active ingredient formulation according to claim 1, wherein at least one known encapsulation material selected from the group which is formed from homo- or heteropolymers of carbohydrates, natural and non-natural amino acids, natural and non-natural nucleic acids, polyamines, polyols, oligo- and polyisoprenes, amides, glucosamines, esters, in particular branched glycerol ester amides, imines, polyphenols, dithiols and phosphodiesters, ethylene glycols, oxymethylene glycoside, acetal units, silicates and carbonates, gum arabic, agar agar, agarose, maltodextrins, alginic acid, alginates, fats, fatty acids, cetyl alcohol, collagen, chitosan, lecithin, gelatin, albumin, shellac, polysaccharides, cyclodextrins, sucrose, waxes, chemically modified celluloses, in particular cellulose esters and ethers, e.g. cellulose acetate, ethylcellulose, hydroxypropylcellulose, hydroxypropylmethylcellulose and carboxymethylcellulose, and also starch derivatives, in particular starch ethers and esters, amino resins, polyacrylates, polyamides, polyvinyl alcohol or polyvinylpyrrolidone, organopolysiloxanes, hyperbranched hydrogels, comb polymers with polyester structure or polyvinylpyrrolidone, and also a polylactide, a polylactide coglycolide or a polycaprolactone is used as additional fraction of encapsulation materials.

7. Encapsulated active ingredient formulation according to claim 1, wherein the active ingredient formulation includes active ingredients selected from the group of amino acid derivatives, such as creatine, vitamins, enzymes or antiinflammatory substances, surfactants, cosmetic oils, pearlescent waxes, stabilizers, antimicrobial active ingredients, antiinflammatory active ingredients, extracts of plant, yeast and algae, synthetic natural substances, vitamins, vitamin derivatives and complexes, amino acids, bioactive lipids, such as cholesterol, ceramides and pseudoceramides, deodorants, antiperspirants, antidandruff agents, UV sun protection factors, antioxidants, preservatives, insect repellants, self-tanning agents, tyrosinase inhibitors, perfume oils, peroxides, peptides, oligopeptides or fragrances.

8. Encapsulated active ingredient formulation according to claim 1, wherein the microparticulate active ingredient formulations have particle sizes between 1 and 1000 μm.

9. Process for the preparation of the encapsulated active ingredient formulation according to claim 1, wherein the particles of the active ingredient formulation are prepared by processes known per se in a temperature range between −30° C. and +150° C. and, in said processes, the system pressure is between 0.1 mbar and 250 bar.

10. Process for the preparation of an encapsulated, microparticulate active ingredient formulation according to claim 9, wherein the particles of the active ingredient formulation are prepared in a temperature range between −30° C. and +150° C. by a combination of at least three processing steps, consisting of at least one stirring step, in which the preferably molten polymer is mixed with a biologically active ingredient, of at least one phase- or particle-separation step and of at least one drying step.

11. (canceled)

12. (canceled)

13. A cosmetic and dermatological formulation for the surface treatment of skin and skin appendages comprising the encapsulated, microparticulate active ingredient formulation according to claim 1.

14. A surface-treated textile comprising the encapsulated, microparticulate active ingredient formulation according to claim 1.
